(19) United States
(12) Patent Application Publication
(10) Pub. No.: US 2023/0265149 A1
(43) Pub. Date: Aug. 24, 2023

(54) ENCAPSULATED CELLS EXPRESSING IL-2 AND USES THEREOF

(71) Applicant: William Marsh Rice University, Houston, TX (US)

(72) Inventors: Omid VEISEH, Houston, TX (US); David ZHANG, Houston, TX (US); Sudip MUKHERJEE, Houston, TX (US); Maria RUOCCO, Houston, TX (US); Michael DOERFERT, Houston, TX (US); Amanda NASH, Houston, TX (US); Samira AGHLARA-FOTOVAT, Houston, TX (US)

(73) Assignee: William Marsh Rice University, Houston, TX (US)

(21) Appl. No.: 18/047,856

(22) Filed: Oct. 19, 2022

Related U.S. Application Data
(60) Provisional application No. 63/257,891, filed on Oct. 20, 2021.

(51) Int. Cl. C07K 14/55; C12N 5/079; C12N 5/00; A61P 35/00

(52) U.S. Cl. CPC C07K 14/55; C12N 5/0621; C12N 5/0012; A61P 35/00

(57) ABSTRACT

The present disclosure relates to implantable constructs (encapsulated cells) designed to deliver antigenic therapeutic reagents, such as IL-2.

### CROSS-REFERENCE TO RELATED APPLICATIONS

The present application claims priority to U.S. Provisional Application No. 63/257,891, filed Oct. 20, 2021, which is hereby incorporated by reference in its entirety.

### STATEMENT REGARDING FEDERALLY FUNDED RESEARCH

This invention was made with government support under Grant No. R01DK120459 awarded by the National Institutes of Health. The government has certain rights in the invention.

### REFERENCE TO A SEQUENCE LISTING

This application contains a Sequence Listing XML, which has been submitted electronically and is hereby incorporated by reference in its entirety. Said XML Sequence Listing, created on Oct. 18, 2022, is named RICEP0095US.xml and is 10,978 bytes in size.

### BACKGROUND

### I. Field

The present disclosure relates to the fields of biology, medicine, bioengineering and medicals devices. More particular, it relates to the development and use of implantable constructs designed to deliver antigenic therapeutic reagents to a subject and protect them from immune responses generated by the host. In particular, the constructs are designed to degrade over time or upon a particular signal, thereby providing control of the length of time the therapeutic agent is delivered to the subject.

### II. Related Art

Advances in biomedical research have led to methods for localized and targeted therapies for the treatment of diseases, such as cancer. However, in many instances, the percentage of patients responsive to these approaches remain modest (Park et al., Sci. Transl. Med. 10(433) 2018).

One approach involves the use of implantable devices to deliver therapeutic agents, however, a fundamental barrier to successful device-based therapies is the inability to deliver a sustained amount of therapeutics that do not have a systemic toxic impact on the subject. Thus, there is a need for identifying new compositions and methods to enhance the delivery, distribution, and/or efficacy of therapeutic agents.

The development of this invention was funded in part by the Cancer Prevention and Research Institute of Texas under Grant No. RR160047.

### SUMMARY

In some embodiments, populations of encapsulated cells comprising an oligonucleotide molecule encoding native human IL-2 are provided. In some embodiments, the oligonucleotide comprises a sequence of SEQ ID NO: 1. In some embodiments, the oligonucleotide encoding native human IL-2 comprises a sequence that is codon-optimized. In some embodiments, the cell produces recombinant native human IL-2 protein. In some embodiments, the recombinant native human IL-2 protein expressed by the cells comprises the amino acid sequence of SEQ ID NO: 2. In some embodiments, the population of cell produces about 0.5 to about 10, about 1 to about 5, or about 2 to about 4 PCD (picograms/cell/day) of native human IL-2. In some embodiments, the cells are encapsulated with a polymeric hydrogel. In some embodiments, the cells remain viable for at least 15, 20, 25, or 28 days. In some embodiments, the encapsulated cells do not proliferate. In some embodiments, the encapsulated cells produced a sustained amount of IL-2 for at least 5, 10, 15, 20, or 24 hours. In some embodiments, the encapsulated cells can produce a sustained amount of IL-2 for up to 30 days.

In some embodiments, pharmaceutical compositions comprising the population of encapsulated cells provided for herein are provided.

In some embodiments, method of treating a tumor, such as a pancreatic tumor, in a subject are provided. In some embodiments, the methods comprise implanting in the intraperitoneal space of the subject a pharmaceutical composition comprising a plurality of encapsulated cells (as provided herein to the subject to treat the cancer.

In some embodiments, methods of treating a tumor, such as a pancreatic tumor, in a subject by generating memory immunity are provided. In some embodiments, the methods comprise implanting a pharmaceutical composition comprising a population of encapsulated cells as provided for herein.

In some embodiments, methods of selectively activating CD8 positive effector T cells are provided. In some embodiments, the methods comprise implanting a pharmaceutical composition comprising a population of encapsulated cells as provided for herein.

In some embodiments, methods of providing systemic treatment to a subject with cancer are provided. In some embodiments, the methods comprise implanting in the intraperitoneal space of the subject a pharmaceutical composition comprising a plurality of encapsulated cells as provided for herein.

In some embodiments, methods of preparing encapsulated cells producing a recombinant protein are provided. In some embodiments, the methods comprise feeding through a coaxial needle a first composition comprising a polymeric hydrogel and a second composition comprising cells to be encapsulated suspended in a polymeric hydrogel to drop into a crosslinking solution to form the encapsulated cells, wherein the crosslinking solution comprises a sugar alcohol, a buffer, a metal salt, and a surfactant.

In some embodiments, populations of encapsulated cells prepared according to a method provided for herein are provided.

In some embodiments, suspensions of encapsulated cells are provided, wherein the suspension comprises a population of encapsulated cells as provided herein, wherein the encapsulated cells are encapsulated by a polymeric hydrogel, and the suspension a crosslinking solution that comprises a sugar alcohol, a buffer, a metal salt, and a surfactant.

In some embodiments, suspensions of encapsulated cells are provided, wherein the suspension comprises a population of encapsulated cells as provided for herein, wherein the encapsulated cells are encapsulated by a polymeric hydrogel, and a storage buffer, such as DMEM/F12 cell culture media.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating certain embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. In vitro T cell Proliferation.

### DETAILED DESCRIPTION

The present disclosure features implantable constructs for delivery of native human IL-2 to a subject in a controlled release manner, and related methods of use thereof. These embodiments will be described below in more detail.

### I. Definitions

“Cell,” as used herein, refers to an individual cell. In an embodiment, a cell is a primary cell or is derived from a cell culture. In an embodiment, a cell is a stem cell or is derived from a stem cell. A cell may be xenogeneic, autologous, or allogeneic. In an embodiment, a cell is engineered (e.g., genetically engineered) or is not engineered (e.g., not genetically engineered). In some embodiments, the cell is an APRE-19 cell. In some embodiments, the cell expresses recombinant native human IL-2 protein. As used herein, the term “recombinant native human IL-2 protein” or “native human IL-2 protein” refers to a protein that comprises the post-translational modifications of IL-2 produced by a cell expressing endogenous IL-2. For example, the heterologous IL-2 produced by the encapsulated cells provided for herein when compared to wild-type IL-2 produced by a cell in the subject has the same or similar post-translational modifications.

“Prevention,” “prevent,” and “preventing” as used herein refers to a treatment that comprises administering or applying a therapy, e.g., administering an implantable construct (e.g., as described herein) comprising a therapeutic agent (e.g., a therapeutic agent described herein) prior to the onset of a disease or condition in order to preclude the physical manifestation of said disease or condition. In some embodiments, “prevention,” “prevent,” and “preventing” require that signs or symptoms of the disease or condition have not yet developed or have not yet been observed. In some embodiments, treatment comprises prevention and in other embodiments it does not. In some embodiments, the prevention is the prevention of the recurrence of a disease, such as a tumor (cancer) after the tumor or cancer has been eradicated by an initial treatment.

“Subject,” as used herein, refers to the recipient of the implantable construct described herein. The subject may include a human and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development (e.g., a male or female of any age group, e.g., a pediatric subject (e.g., infant, child, adolescent) or adult subject (e.g., young adult, middle-aged adult, or senior adult). A non-human animal may be a transgenic animal.

“Treatment,” “treat,” and “treating,” as used herein, refer to reversing, alleviating, delaying the onset of, or inhibiting the progress of one or more of a symptom, manifestation, or underlying cause of a disease or condition. (e.g., as described herein), e.g., by administering or applying a therapy, e.g., administering an implantable construct comprising a therapeutic agent (e.g., a therapeutic agent described herein). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease or condition. In an embodiment, treating comprises reducing, reversing, alleviating, reducing, or delaying the onset of, an underlying cause of a disease or condition. In some embodiments, “treatment,” “treat,” and “treating” require that signs or symptoms of the disease or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

### B. Cells

Implantable constructs described herein may contain a cell, for example, an engineered cell. A cell be derived from any mammalian organ or tissue, including the brain, nerves, ganglia, spine, eye, heart, liver, kidney, lung, spleen, bone, thymus, lymphatic system, skin, muscle, pancreas, stomach, intestine, blood, ovary, uterus, or testes. In some embodiments, the cell is a APRE-19 cell.

A cell may be derived from a donor (e.g., an allogeneic cell), derived from a subject (e.g., an autologous cell), or from another species (e.g., a xenogeneic cell). In an embodiment, a cell can be grown in cell culture, or prepared from an established cell culture line, or derived from a donor (e.g., a living donor or a cadaver). In an embodiment, a cell is genetically engineered. In another embodiment, a cell is not genetically engineered. A cell may include a stem cell, such as a reprogrammed stem cell, or an induced pluripotent cell. Exemplary cells include mesenchymal stem cells (MSCs), fibroblasts (e.g., primary fibroblasts). HEK cells (e.g., HEK293T), Jurkat cells, HeLa cells, retinal pigment epithelial (RPE) cells, HUVEC cells, NIH3T3 cells, CHO-K1 cells, COS-1 cells, COS-7 cells, PC-3 cells, HCT 116 cells, A549MCF-7 cells, HuH-7 cells, U-2 OS cells, HepG2 cells, Neuro-2a cells, and SF9 cells. In an embodiment, a cell for use in an implantable construct is an RPE cell.

A cell included in an implantable construct may produce or secrete a therapeutic agent, such as native human IL-2. In an embodiment, a cell included in an implantable construct may produce or secrete a single type of therapeutic agent or a plurality of therapeutic agents. In an embodiment, an implantable construct may comprise a cell that is transduced or transfected with a nucleic acid (e.g., a vector) comprising an expression sequence of a therapeutic agent. For example, a cell may be transduced or transfected with a lentivirus. A nucleic acid introduced into a cell (e.g., by transduction or transfection) may be incorporated into a nucleic acid delivery system, such as a plasmid, or may be delivered directly. In an embodiment, a nucleic acid introduced into a cell (e.g., as part of a plasmid) may include a region to enhance expression of the therapeutic agent and/or to direct targeting or secretion, for example, a promoter sequence, an activator sequence, or a cell-signaling peptide, or a cell export peptide. Exemplary promoters include EF-1a, CMV, Ubc, hPGK, VMD2, and CAG. Exemplary activators include the TET1 catalytic domain, P300 core, VPR, rTETR, Cas9 (e.g., from S. pyogenes or S. aureus), and Cpf1 (e.g., from L. bacterium).

An implantable construct described herein may comprise a cell or a plurality of cells. In the case of a plurality of cells, the concentration and total cell number may be varied depending on a number of factors, such as cell type, implantation location, and expected lifetime of the implantable construct. In an embodiment, the total number of cells included in an implantable construct is greater than about 2, 4, 6, 8, 10, 20, 30, 40, 50, 75, 100, 200, 250, 500, 750, 1000, 1500, 2000, 5000, 10000, or more. In an embodiment, the total number of cells included in an implantable construct is greater than about 1.0×10², 1.0×10³, 1.0×10⁴, 1.0×10⁵, 1.0×10⁶, 1.0×10⁷, 1.0×10⁸, 1.0×10⁹, 1.0×10¹⁰, or more. In an embodiment, the total number of cells included in an implantable construct is less than about than about 10000, 5000, 2500, 2000, 1500, 1000, 750, 500, 250, 200, 100, 75, 50, 40, 30, 20, 10, 8, 6, 4, 2, or less. In an embodiment, the total number of cells included in an implantable construct is less than about 1.0×10¹⁰, 1.0×10⁹, 1.0×10⁸, 1.0×10⁷, 1.0×10⁶, 1.0×10⁵, 1.0×10⁴, 1.0×10³, 1.0×10², or less. In an embodiment, a plurality of cells is present as an aggregate. In an embodiment, a plurality of cells is present as a cell dispersion.

Specific features of a cell contained within an implantable construct may be determined, e.g., prior to and/or after incorporation into the implantable construct. For example, cell viability, cell density, or cell expression level may be assessed. In an embodiment, cell viability, cell density, and cell expression level may be determined using standard techniques, such as cell microscopy, fluorescence microscopy, histology, or biochemical assay.

### C. Therapeutic Agents

An implantable construct described herein may contain a therapeutic agent, for example, produced or secreted by a cell, such as native human IL-2. A therapeutic agent may include a nucleic acid encoding the protein (e.g., an RNA, a DNA, or an oligonucleotide), a protein (e.g., an antibody, enzyme, cytokine, hormone, receptor) that is secreted from the cell, and the like. In an embodiment, the implantable construct comprises a cell or a plurality of cells that are genetically engineered to produce or secrete a therapeutic agent.

In some embodiments, native human IL-2 refers to a protein encoded by a nucleic acid sequence comprising

(SEQ ID NO: 1)
ATGTACAGGATGCAACTCCTGTCTTGCATTGCACTAAGTCTTGCACTTG
TCACAAACAGTGCACCTACTTCAAGTTCTACAAAGAAAACACAGCTACA
ACTGGAGCATTTACTGCTGGATTTACAGATGATTTTGAATGGAATTAAT
AATTACAAGAATCCCAAACTCACCAGGATGCTCACATTTAAGTTTTACA
TGCCCAAGAAGGCCACAGAACTGAAACATCTTCAGTGTCTAGAAGAAGA
ACTCAAACCTCTGGAGGAAGTGCTAAATTTAGCTCAAAGCAAAAACTTT
CACTTAAGACCCAGGGACTTAATCAGCAATATCAACGTAATAGTTCTGG
AACTAAAGGGATCTGAAACAACATTCATGTGTGAATATGCTGATGAGAC
AGCAACCATTGTAGAATTTCTGAACAGATGGATTACCTTTTGTCAAAGC
ATCATCTCAACACTGACTTGA.

In some embodiments, the nucleic acid coding sequence encoding native human IL-2 is codon-optimized. In some embodiments, the nucleic acid coding sequence encoding native human IL-2 is codon-optimized for expression in a mammalian cell. The codon optimized sequence may be generated using a commercially available algorithm, e.g., GeneOptimizer (ThermoFisher Scientific), OptimumGene™ (GenScript, Piscataway, N.J. USA), GeneGPS® (ATUM, Newark, Calif. USA), Java Codon Adaptation Tool (JCat, http://www.jcat.de, Grote, A. et al., Nucleic Acids Research, Vol 33, Issue suppl_2, pp. W526-W531 (2005), IDT Codon Optimization Tool (Integrated DNA Technologies), VectorBuilder Codon Optimization tool (VectorBuilder Inc.), Codon Optimization OnLine (COOL, http://bioinfo.bti.a-star.edu.sg/COOL/; Chin J. X., et al., Bioinformatics, Vol 30, Issue 15, p.2210-2212 (2014)), or ExpOptimizer (NovoPro, Shanghai, China). Examples of codon-optimized nucleic acid coding sequences encoding native human IL-2 comprise, but are not limited to:

(SEQ ID NO: 3)
ATGTACCGGATGCAGCTGCTGTCCTGCATCGCACTGTCCCTCGCCCTGG
TGACAAATTCTGCCCCCACCTCCTCCAGCACAAAAAAGACCCAGTTGCA
GCTGGAGCACCTGCTGCTGGATCTGCAGATGATCCTGAATGGCATCAAT
AACTACAAAAACCCTAAACTGACCAGAATGCTGACCTTTAAATTTTACA
TGCCTAAAAAGGCAACCGAGCTGAAGCACCTGCAGTGCCTGGAAGAGGA
ACTGAAGCCCCTGGAGGAGGTGCTGAACCTGGCCCAGAGCAAGAACTTT
CACCTGCGGCCCCGCGACCTGATCAGCAACATCAACGTGATCGTGCTGG
AGCTGAAGGGCAGTGAAACCACATTCATGTGCGAGTACGCCGACGAGAC
CGCCACAATCGTGGAGTTCCTGAACAGATGGATCACATTCTGTCAGTCC
ATCATTAGCACACTGACCTAA;
(SEQ ID NO: 4)
ATGTACCGCATGCAGCTGCTGAGCTGCATCGCCCTGAGCCTGGCCCTGG
TGACCAACAGCGCCCCCACCAGCAGCAGCACCAAGAAGACCCAGCTGCA
GCTGGAGCACCTGCTGCTGGACCTGCAGATGATCCTGAACGGCATCAAC
AACTACAAGAACCCCAAGCTGACCCGCATGCTGACCTTCAAGTTCTACA
TGCCCAAGAAGGCCACCGAGCTGAAGCACCTGCAGTGCCTGGAGGAGGA
GCTGAAGCCCCTGGAGGAGGTGCTGAACCTGGCCCAGAGCAAGAACTTC
CACCTGCGCCCCCGCGACCTGATCAGCAACATCAACGTGATCGTGCTGG
AGCTGAAGGGCAGCGAGACCACCTTCATGTGCGAGTACGCCGACGAGAC
CGCCACCATCGTGGAGTTCCTGAACCGCTGGATCACCTTCTGCCAGAGC
ATCATCAGCACCCTGACCTAA;
(SEQ ID NO: 5)
ATGTATAGGATGCAGCTGCTCTCTTGTATCGCGTTGTCTCTGGCTTTGG
TGACTAACTCAGCTCCCACGTCCAGCAGTACCAAAAAGACCCAGCTGCA
GCTGGAACATCTTCTGTTGGATCTGCAAATGATACTGAATGGGATCAAC
AACTATAAAAACCCAAAACTGACTAGAATGCTGACTTTCAAGTTCTACA
TGCCTAAAAAGGCAACAGAATTGAAGCACCTTCAGTGCCTGGAGGAGGA
GCTTAAGCCCCTGGAGGAGGTGCTGAATCTGGCCCAAAGTAAGAATTTT
CATCTGCGACCCAGGGATCTGATCAGTAATATCAATGTGATCGTCCTGG
AGCTGAAGGGCAGTGAGACCACGTTTATGTGTGAATACGCAGACGAAAC
CGCCACTATCGTTGAATTCTTGAACAGGTGGATCACCTTTTGTCAGAGT
ATCATCAGCACCCTCACT;
or
(SEQ ID NO: 6)
ATGTACAGAATGCAGCTGCTGAGCTGCATCGCCCTGAGCCTGGCCCTGG
TGACCAACAGCGCCCCCACAAGCAGCAGCACCAAGAAGACACAGCTGCA
GCTGGAGCACCTGCTGCTGGACCTGCAGATGATCCTGAACGGCATCAAC
AACTACAAGAACCCCAAGCTGACAAGAATGCTGACCTTCAAGTTCTACA
TGCCCAAGAAGGCCACCGAGCTGAAGCACCTGCAGTGCCTGGAGGAGGA
GCTGAAGCCCCTGGAAGAGGTGCTGAACCTGGCTCAGAGCAAGAACTTC
CACCTGAGACCTAGAGACCTGATCAGCAACATCAACGTGATCGTGCTGG
AGCTGAAGGGCAGCGAGACCACCTTCATGTGCGAGTACGCCGACGAGAC
CGCCACCATCGTGGAGTTCCTGAACAGATGGATCACCTTCTGTCAGAGC
ATCATCAGCACCCTGACCTGA.

In some embodiments, the codon-optimized nucleic acid coding sequence encoding native human IL-2 comprise the nucleic acid sequence as set forth in SEQ ID NO: 3-6. In some embodiments, the codon-optimized nucleic acid coding sequence encoding native human IL-2 comprise the nucleic acid sequence as set forth in SEQ ID NO: 3. In some embodiments, the codon-optimized nucleic acid coding sequence encoding native human IL-2 comprise the nucleic acid sequence having at least 90%, 92%, 93%, 94%, 95%, 96%, 97%, 98%, 99%, or 100% identity to the sequence of SEQ ID NO: 3-6. In some embodiments, the codon-optimized nucleic acid coding sequence encoding native human IL-2 comprise the nucleic acid sequence having at least 90%, 92%, 93%, 94%, 95%, 96%, 97%, 98%, 99%, or 100% identity to the sequence of SEQ ID NO: 3.

In some embodiments, the native human protein produced by the cell is formed from the formed from an amino acid sequence of:

(SEQ ID NO: 2)
MYRMQLLSCIALSLALVTNSAPTSSSTKKTQLQLEHLLLDLQMILNGIN
NYKNPKLTRMLTFKFYMPKKATELKHLQCLEEELKPLEEVLNLAQSKNF
HLRPRDLISNINVIVLELKGSETTFMCEYADETATIVEFLNRWITFCQS
IISTLT.

Without being bound by any particular theory, the native IL-2 produced by the cells comprising the nucleic acid sequence of SEQ ID NO: 1 differs from recombinant IL-2 produced from bacteria or other non-eukaryotic cells due to differences in post-translational modification. Thus, the cells expressing the native human IL-2 is superior because it has superior potency. This is described herein in the Examples section. In some embodiments, the IL-2 is a IL-2 mutein or a modified IL-2 molecule, fusion proteins or antibodies that act on the IL-2 pathway. In some embodiments, the IL-2 is a pegylated IL-2 molecule. In some embodiments, the pegylated IL-2 molecule has a wild-type sequence. In some embodiments, the pegylated IL-2 has a mutant IL-2 sequence. In some embodiments, the capsules or cells are used to produce NKTR-214 (pegylated IL-2; Clin Cancer Res Feb. 1 2016 (22) (3) 680-690; DOI: 10.1158/1078-0432.CCR-15-1631, which is hereby incorporated by reference in its entirety), THOR-707 (SAR444245; Annals of Oncology, Volume 30, Supplement 5, October 2019, Page v501, which is hereby incorporated by reference in its entirety), ALKS 4230 (J Immunother Cancer. 2020 April; 8(1):e000673. doi: 10.1136/jitc-2020-000673, Nemvaleukin Alfa), TransCon IL-2 β/γ, BNT151, BNT153, CLN-617, CUE-101, CUE-102, CUE-103, Anktiva® (N-803), KY1043, MDNA11, NL-201, SO-C101, RO6874281, Simlukafusp Alfa, RG7461, WTX-124, WTX-330, XTX202, or XTX401 or any combination thereof.

The cell can also be modified to produce or secrete an additional protein or molecule in addition to native human IL-2. In some embodiments, the additional protein or molecule is a IL-2 mutein or a modified IL-2 molecule, fusion proteins or antibodies that act on the IL-2 pathway. In some embodiments, the IL-2 is a pegylated IL-2 molecule. In some embodiments, the pegylated IL-2 molecule has a wild-type sequence. In some embodiments, the pegylated IL-2 has a mutant IL-2 sequence. In some embodiments, the additional protein or molecule is selected from NKTR-214, THOR-707, ALKS 4230, Nemvaleukin Alfa, TransCon IL-2 β/γ, BNT151, BNT153, CLN-617, CUE-101, CUE-102, CUE-103, Anktiva® (N-803), KY1043, MDNA11, NL-201, SO-C101, RO6874281, Simlukafusp Alfa, RG7461, WTX-124, WTX-330, XTX202, or XTX401, or any combination thereof. The additional protein may be of any size, e.g., greater than about 100 Da, 200 Da, 250 Da, 500 Da, 750 Da, 1 KDa, 1.5 kDa, 2 kDa, 2.5 kDa, 3 kDa, 4 kDa, 5 kDa, 6 kDa, 7 kDa, 8 kDa, 9 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 95 kDa, 100 kDa, 125 kDa, 150 kDa, 200 kDa, 200 kDa, 250 kDa, 300 kDa, 400 kDa, 500 kDa, 600 kDa, 700 kDa, 800 Da, 900 kDa, or more. In an embodiment, the protein is composed of a single subunit or multiple subunits (e.g., a dimer, trimer, tetramer, etc.). A protein produced or secreted by a cell may be modified, for example, by glycosylation, methylation, or other known natural or synthetic protein modification. A protein may be produced or secreted as a pre-protein or in an inactive form and may require further modification to convert it into an active form.

Proteins produced or secreted by a cell may be include antibodies or antibody fragments, for example, an Fc region or variable region of an antibody. Exemplary antibodies include anti-PD-1, anti-PD-L1, anti-CTLA4, anti-TNFα, and anti-VEGF antibodies. An antibody may be monoclonal or polyclonal. Other exemplary proteins include a lipoprotein, an adhesion protein, hemoglobin, enzymes, proenkephalin, a growth factor (e.g., EGF, IGF-1, VEGF alpha, HGF, TGF beta, bFGF), or a cytokine.

A protein produced or secreted by a cell may also include a hormone. Exemplary hormones include growth hormone, growth hormone releasing hormone, prolactin, lutenizing hormone (LH), anti-diuretic hormone (ADH), oxytocin, thyroid stimulating hormone (TSH), thyrotropin-release hormone (TRH), adrenocorticotropic hormone (ACTH), follicle-stimulating hormone (FSH), thyroxine, calcitonin, parathyroid hormone, aldosterone, cortisol, epinephrine, glucagon, insulin, estrogen, progesterone, and testosterone.

A protein produced or secreted by a cell may include other cytokines. A cytokine may be a pro-inflammatory cytokine or an anti-inflammatory cytokine. Example of cytokines include IL-1, IL-1α, IL-1β, IL-1RA, IL-4, IL-5, IL-6, IL-7, IL-8, IL-9, IL-10, IL-11, IL-12, IL-12a, IL-12b, IL-13, IL-14, IL-15, IL-16, IL-17, G-CSF, GM-CSF, IL-20, IL-23, IFN-α, IFN-β, IFN-γ, CD154, LT-β, CD70, CD153, CD178, TRAIL, TNF-α, TNF-β, SCF, M-CSF, MSP, 4-1BBL, LIF, OSM, and others. For example, a cytokine may include any cytokine described in M. J. Cameron and D. J. Kelvin, Cytokines, Chemokines, and Their Receptors (2013), Landes Biosciences, which is incorporated herein by reference in its entirety.

An provided for herein implantable construct may comprise a cell expressing a single type of therapeutic agent, e.g., a single protein or nucleic acid, or may express more than one type of therapeutic agent, e.g., a plurality of proteins or nucleic acids. In an embodiment, an implantable construct comprises a cell expressing two types of therapeutic agents (e.g., two types of proteins or nucleic acids). In an embodiment, an implantable construct comprises a cell expressing three types of therapeutic agents (e.g., three types of proteins or nucleic acids). In an embodiment, an implantable construct comprises a cell expressing four types of therapeutic agents (e.g., four types of proteins or nucleic acids).

In an embodiment, an implantable construct comprises a cell expressing a single type of nucleic acid (e.g., DNA or RNA) or may express more than one type of nucleic acid, e.g., a plurality of nucleic acid (e.g., DNA or RNA). In an embodiment, an implantable construct comprises a cell expressing two types of nucleic acids (e.g., DNA or RNA). In an embodiment, an implantable construct comprises a cell expressing three types of nucleic acids (e.g., DNA or RNA). In an embodiment, an implantable construct comprises a cell expressing four types of nucleic acids (e.g., DNA or RNA).

In an embodiment, an implantable construct comprises a cell expressing a single type of protein, or may express more than one type of protein, e.g., a plurality of proteins. In an embodiment, an implantable construct comprises a cell expressing two types of proteins. In an embodiment, an implantable construct comprises a cell expressing three types of proteins. In an embodiment, an implantable construct comprises a cell expressing four types of proteins.

In an embodiment, an implantable construct comprises a cell expressing a single type of enzyme, or may express more than one type of enzyme, e.g., a plurality of enzymes. In an embodiment, an implantable construct comprises a cell expressing two types of enzymes. In an embodiment, an implantable construct comprises a cell expressing three types of enzymes. In an embodiment, an implantable construct comprises a cell expressing four types of enzymes.

In an embodiment, an implantable construct comprises a cell expressing a single type of antibody or antibody fragment or may express more than one type of antibody or antibody fragment, e.g., a plurality of antibodies or antibody fragments. In an embodiment, an implantable construct comprises a cell expressing two types of antibodies or antibody fragments. In an embodiment, an implantable construct comprises a cell expressing three types of antibodies or antibody fragments. In an embodiment, an implantable construct comprises a cell expressing four types of antibodies or antibody fragments.

In an embodiment, an implantable construct comprises a cell expressing a single type of hormone, or may express more than one type of hormone, e.g., a plurality of hormones. In an embodiment, an implantable construct comprises a cell expressing two types of hormones. In an embodiment, an implantable construct comprises a cell expressing three types of hormones. In an embodiment, an implantable construct comprises a cell expressing four types of hormones.

In an embodiment, an implantable construct comprises a cell expressing a single type of enzyme, or may express more than one type of enzyme, e.g., a plurality of enzymes. In an embodiment, an implantable construct comprises a cell expressing two types of enzymes. In an embodiment, an implantable construct comprises a cell expressing three types of enzymes. In an embodiment, an implantable construct comprises a cell expressing four types of enzymes.

In an embodiment, an implantable construct comprises a cell expressing a single type of cytokine or may express more than one type of cytokine, e.g., a plurality of cytokines. In an embodiment, an implantable construct comprises a cell expressing two types of cytokines. In an embodiment, an implantable construct comprises a cell expressing three types of cytokines. In an embodiment, an implantable construct comprises a cell expressing four types of cytokines.

### D. Features of Implantable Constructs

The implantable construct described herein may take any suitable shape or morphology. For example, an implantable construct may be a sphere, spheroid, tube, cord, string, ellipsoid, disk, cylinder, sheet, torus, cube, stadiumoid, cone, pyramid, triangle, rectangle, square, or rod. An implantable construct may comprise a curved or flat section. In an embodiment, an implantable construct may be prepared through the use of a mold, resulting in a custom shape.

The implantable construct may vary in size, depending, for example, on the use or site of implantation. For example, an implantable construct may have a mean diameter or size greater than 0.1 mm, e.g., greater than 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, or more. In an embodiment, an implantable construct may have a section or region with a mean diameter or size greater than 0.1 mm, e.g., greater than 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, or more. In an embodiment, an implantable construct may have a mean diameter or size less than 1 cm, e.g., less 50 mm, 40 mm, 30 mm, 20 mm, 10 mm, 7.5 mm, 5 mm, 2.5 mm, 1 mm, 0.5 mm, or smaller. In an embodiment, an implantable construct may have a section or region with a mean diameter or size less than 1 cm, e.g., less 50 mm, 40 mm, 30 mm, 20 mm, 10 mm, 7.5 mm, 5 mm, 2.5 mm, 1 mm, 0.5 mm, or smaller.

An implantable construct comprises at least one zone capable of preventing exposure of an enclosed antigenic or therapeutic agent from the outside milieu, e.g., a host effector cell or tissue. In an embodiment, the implantable construct comprises an inner zone (IZ). In an embodiment, the implantable construct comprises an outer zone (OZ). In an embodiment, either the inner zone (IZ) or outer zone (OZ) may be erodible or degradable. In an embodiment, the inner zone (IZ) is erodible or degradable. In an embodiment, the outer zone (OZ) is erodible or degradable. In an embodiment, the implantable construct comprises both an inner zone (IZ) and an outer zone (OZ), either of which may be erodible or degradable. In an embodiment, the implantable construct comprises both an inner zone (IZ) and an outer zone (OZ), wherein the outer zone is erodible or degradable. In an embodiment, the implantable construct comprises both an inner zone (IZ) and an outer zone (OZ), wherein the inner zone is erodible or degradable. The thickness of either of the zone, e.g., either the inner zone or outer zone, may be correlated with the length or duration of a “shielded” phase, in which the encapsulated antigenic or therapeutic agent is protected or shielded from the outside milieu, e.g., a host effector cell or tissue.

The zone (e.g., the inner zone or outer zone) of the implantable construct may comprise a degradable entity, e.g., an entity capable of degradation. A degradable entity may comprise an enzyme cleavage site, a photolabile site, a pH-sensitive site, or other labile region that can be eroded or comprised over time. In an embodiment, the degradable entity is preferentially degraded upon exposure to a first condition (e.g., exposure to a first milieu, e.g., a first pH or first enzyme) relative to a second condition (e.g., exposure to a second milieu, e.g., a second pH or second enzyme). In one embodiment, the degradable entity is degraded at least 2, 5, 10, 20, 30, 40, 50, 60, 70, 80, or 100 times faster upon exposure to a first condition relative to a second condition. In an embodiment, the degradable entity is an enzyme cleavage site, e.g., a proteolytic site. In an embodiment, the degradable entity is a polymer (e.g., a synthetic polymer or a naturally occurring polymer, e.g., a peptide or polysaccharide). In an embodiment, the degradable entity is a substrate for an endogenous host component, e.g., a degradative enzyme, e.g., a remodeling enzyme, e.g., a collagenase or metalloprotease. In an embodiment, the degradable entity comprises a cleavable linker or cleavable segment embedded in a polymer.

In an embodiment, an implantable construct comprises a pore or opening to permit passage of an object, such as a small molecule (e.g., nutrients or waste), a protein, or a nucleic acid. For example, a pore in or on an implantable construct may be greater than 0.1 nm and less than 10 μm. In an embodiment, the implantable construct comprises a pore or opening with a size range of 0.1 μm to 10 μm, 0.1 μm to 9 μm, 0.1 μm to 8 μm, 0.1 μm to 7 μm, 0.1 μm to 6 μm, 0.1 μm to 5 μm, 0.1 μm to 4 μm, 0.1 μm to 3 μm, 0.1 μm to 2 μm.

An implantable construct described herein may comprise a chemical modification in or on any enclosed material. Exemplary chemical modifications include small molecules, peptides, proteins, nucleic acids, lipids, or oligosaccharides. The implantable construct may comprise at least 0.5%, 1%, 2%, 3%, 4%, 5%, 7.5%, 10%, 15%, 20%, 30%, 40%, 50%, 60%, 70%, 80% or more of a material that is chemically modified, e.g., with a chemical modification described herein. An implantable construct may be partially coated with a chemical modification, e.g., at least about 5%, 10%, 15%, 20%, 25%, 30%, 35%, 40%, 45%, 50%, 55%, 60%, 65%, 70%, 75%, 80%, 85%, 90%, 95%, 99%, or 99.9% coated with a chemical modification.

In an embodiment, the implantable construct is formulated such that the duration of release of the antigenic and/or therapeutic agent is tunable. For example, an implantable construct may be configured in a certain manner to release a specific amount of an antigenic or therapeutic agent over time, e.g., in a sustained or controlled manner. In an embodiment, the implantable construct comprises a zone (e.g., an inner zone or an outer zone) that is degradable, and this controls the duration of therapeutic release from the construct by gradually ceasing immunoprotection of encapsulated cells or causing gradual release of the antigenic agent. In an embodiment, the implantable construct is configured such that the level of release of an antigenic or therapeutic agent is sufficient to modulate the ratio of a host effector cell, e.g., a host T cell. In an embodiment, the implantable construct is configured such that the level of release of an antigenic or therapeutic agent is sufficient to activate a host cell (e.g., a host T effector cell or a host NK cell) or increase the level of certain host cells (e.g., host T effector cells or host NK cells). In an embodiment, the implantable construct is configured such that the level of release of an antigenic or therapeutic agent is not sufficient to activate a host regulator cell (e.g., a host T regulator cell) or increase the level of host regulator cells (e.g., host T regulator cells).

In some embodiments, the implantable construct comprises a zone that is targeted by the natural foreign body response (FBR) of a host or subject, e.g., over a period of time. In an embodiment, the implantable construct is coated with fibrotic overgrowth upon administration to a subject, e.g., over a period of time. Fibrotic overgrowth on the surface of the implantable construct may lead to a decrease in function of the implantable construct. For example, a decrease in function may comprise a reduction in the release of an antigenic or therapeutic agent over time, a decrease in pore size, or a decrease in the diffusion rate of oxygen and other key nutrients to the encapsulated cells, leading to cell death. In an embodiment, the rate of fibrotic overgrowth may be tuned to design a dosing regimen. For example, the fibrotic overgrowth on the surface of an implantable construct may result in a decrease in function of the implantable construct about 6 hours, 12 hours, 18 hours, 1 day, 2 days, 3 days, 4 days, 5 days, 6 days, 7 days, 8 days, 9 days, 10 days, 11 days, 12 days, 13 days, 2 weeks, 2.5 weeks, 3 weeks, 4 weeks, or 6 weeks after administration (e.g., injection or implantation) to a subject.

In some embodiments, the implantable construct is chemically modified with a specific density of modifications. The specific density of chemical modifications may be described as the average number of attached chemical modifications per given area. For example, the density of a chemical modification on or in an implantable construct may be 0.01, 0.1, 0.5, 1, 5, 10, 15, 20, 50, 75, 100, 200, 400, 500, 750, 1,000, 2,500, or 5,000 chemical modifications per square μm or square mm.

An implantable construct may be formulated or configured for implantation in any organ, tissue, cell, or part of a subject. For example, the implantable construct may be implanted or disposed into the intraperitoneal space of a subject. An implantable construct may be implanted in or disposed on a tumor or other growth in a subject, or be implanted in or disposed about 0.1 mm, 0.5 mm, 1 mm, 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, 1 cm, 5, cm, 10 cm, or further from a tumor or other growth in a subject. An implantable construct may be configured for implantation, or implanted, or disposed on or in the skin, a mucosal surface, a body cavity, the central nervous system (e.g., the brain or spinal cord), an organ (e.g., the heart, eye, liver, kidney, spleen, lung, ovary, breast, uterus), the lymphatic system, vasculature, oral cavity, nasal cavity, gastrointestinal tract, bone, muscle, adipose tissue, skin, or other area.

An implantable construct may be formulated for use for any period of time. For example, an implantable construct may be used for 1 hour, 2 hours, 4 hours, 6 hours, 12 hours, 1 day, 36 hours, 2 days, 3 days, 4 days, 5 days, 6 days, 1 week, 2 weeks, 3 weeks, 4 weeks, 5 weeks, 6 weeks, 2 months, 3 months, 4 months, 5 months, 6 months, 1 year, or longer. An implantable construct can be configured for limited exposure (e.g., less than 2 days, e.g., less than 2 days, 1 day, 24 hours, 20 hours, 16 hours, 12 hours, 10 hours, 8 hours, 6 hours, 5 hours, 4 hours, 3 hours, 2 hours, 1 hour or less). A implantable construct can be configured for prolonged exposure (e.g., at least 2 days, 3 days, 4 days, 5 days, 6 days, 7 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more). An implantable construct can be configured for permanent exposure (e.g., at least 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more).

In some embodiments, the degradable zone comprises a polymeric hydrogel, such as but not limited to chitosan, cellulose, hyaluronic acid, or alginate. In some embodiments, the alginate is SLG20.

Accordingly, in some embodiments, a population of encapsulated cells comprising an oligonucleotide molecule encoding native human IL-2 are provided. In some embodiments, the oligonucleotide encoding native human IL-2 comprises a sequence of SEQ ID NO: 1. In some embodiments, the cell produces recombinant native human IL-2 protein. In some embodiments, the recombinant native human IL-2 protein expressed by the cells is formed from an amino acid sequence of (SEQ ID NO: 2). In some embodiments, the population of encapsulated cells produces about 1 to about 10, about 1 to about 5, or about 2 to about 4 PCD (picograms/cell/day) of native human IL-2. As provided herein, the cell can be any type of suitable cell, such as ARPE-19 cells.

In some embodiments, the cells in the encapsulated cells, which can also be referred to as the implantable construct, remain viable for at least 15, 20, 25, or 28 days. As used herein, the term, “viable” refers to a cell being able to produce IL-2 over this time period. In some embodiments, a viable cell is not a cell that is dividing. A cell can still be viable even if it is not dividing to expand the number of cells. In some embodiments, the encapsulated cells do not proliferate. Without being bound to any particular theory, the cells do not proliferate once encapsulated due to contact inhibition. In some embodiments, the encapsulated cells produced a sustained amount of IL-2 for at least 5, 10, 15, 20, or 24 hours In some embodiments, the encapsulated cells can produce a sustained amount of IL-2 for up to 30 days. Also provided for herein are pharmaceutical compositions comprising the encapsulated cells.

### E. Methods of Treatment

Described herein are methods of treatment or uses of encapsulated cells for the preparation of a pharmaceutical composition (or medicament) for the treatment of tumors or a disease.

In some embodiments, the disease is a proliferative disease. In an embodiment, the proliferative disease is cancer. A cancer may be an epithelial, mesenchymal, or hematological malignancy. A cancer includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). In an embodiment, the cancer is a solid tumor (e.g., carcinoid, carcinoma or sarcoma), a soft tissue tumor (e.g., a heme malignancy), or a metastatic lesion, e.g., a metastatic lesion of any of the cancers disclosed herein. In an embodiment, the cancer is a fibrotic or desmoplastic solid tumor. In some embodiments, the tumor is a pancreatic tumor.

Accordingly, in some embodiments, methods of treating a tumor, such as a pancreatic tumor, in a subject, are provided. In some embodiments, the methods comprise implanting in the intraperitoneal space of the subject a pharmaceutical composition comprising a plurality of a population of encapsulated cells (e.g., a capsule) as provided for herein to treat the cancer.

A method of providing systemic treatment to a subject with cancer, the method comprising implanting in the intraperitoneal space of the subject a pharmaceutical composition comprising a plurality of a the population of encapsulated cells (e.g., a capsule) as provided for herein, whereby the pharmaceutical composition stimulates the activation of immune cells in the intraperitoneal space and the activated immune cells migrate to a region of the subject that is distal to the intraperitoneal space to treat the cancer systemically in the subject.

In some embodiments, methods of providing systemic treatment to a subject with cancer are provided. In some embodiments, the methods comprise implanting in the intraperitoneal space of the subject a pharmaceutical composition comprising a plurality of a the population of encapsulated cells (e.g., a capsules) as provided herein. In some embodiments, the pharmaceutical composition activates immune cells in the IP space. In some embodiments, the activated immune cells migrate out of (away from) the intraperitoneal space to treat the cancer in the subject at a site that is not in the IP space. In some embodiments, the activated immune cells migrate out of (away from) the intraperitoneal space to treat the cancer in the subject at a site that is distal from the IP space. In some embodiments, the site is another organ or tissue, such as pancreas, breast, brain, lungs, bone, or as otherwise provided for herein. Without being bound to any particular theory, because the compositions provided for herein can deliver native IL-2 that is produced by the cells in a localized space, the negative effects of IL-2 that has been previously delivered systematically can be reduced or eliminated. Thus, the positive, or therapeutic effect, of IL-2 can be delivered to the subject without producing, or by reducing, the systemic side effects seen with the systemic administration of IL-2. Accordingly, in some embodiments, the subject has fewer side effects as compared to a subject that is administered a pharmaceutical composition systemically, such as intravenously administered IL-2 or intravenously administered compositions provided for herein. In some embodiments, the activated immune cells are CD8 positive effector T cells. In some embodiments, the effector T cells are selectively activated and expanded at least 1, 2, 3, 4, or 5 times as compared to Tregs in the intraperitoneal space. In some embodiments, the effector T cells are selectively activated and expanded at least 1, 2, 3, 4, or 5 times as compared to Tregs systemically.

In some embodiments, the methods comprise delivering a concentration of native human IL-2 in the intraperitoneal space at day 5 post implantation that is at least 5000 pg/ml, 10000 pg/ml, 15000 pg/ml, 20000 pg/ml, 50000 pg/ml, 100000 pg/ml, or 150000 pg/ml.

In some embodiments, the concentration of the native human IL-2 in the intraperitoneal space is at least 100× greater than the concentration of the native human IL-2 in the blood of the subject after implantation. In some embodiments, the concentration of the native human IL-2 in the intraperitoneal space is at about 150 to 200 times greater than the concentration of the native human IL-2 in the blood of the subject after implantation. In some embodiments, the concentration is determined at, or at least, 1 day, 2 day, 3 day, 4 day, or 5 days post implantation into the intraperitoneal space. In some embodiments, the concentration of the recombinant native human IL-2 in the blood of the subject is substantially undetectable 5 days after implantation. In some embodiments, the concentration of the recombinant native human IL-2 in the blood of the subject is substantially undetectable 5 days after implantation and is at least 5000 pg/ml, 10000 pg/ml, 15000 pg/ml, 20000 pg/ml, 50000 pg/ml, 100000 pg/ml, or 150000 pg/ml in the intraperitoneal space of the subject. In some embodiments, the recombinant native IL-2 protein is detectable in the intraperitoneal space of the subject at least 1, 4, 7, 14, 21, or 30 days post implantation.

In some embodiments, the subject is administered (e.g., implanted) about 0.01 μg/kg/day to about 20 μg/kg/day, about 0.1 μg/kg/day to about 20 μg/kg/day, about 1 μg/kg/day to about 20 μg/kg/day, about 2 μg/kg/day to about 20 μg/kg/day, about 5 μg/kg/day to about 20 μg/kg/day, about 7.5 to about 20 μg/kg/day, about 9 μg/kg/day to about 20 μg/kg/day, about 10 μg/kg/day to about 20 μg/kg/day, about 11 μg/kg/day to about 20 μg/kg/day, about 12 μg/kg/day to about 20 μg/kg/day, about 13 μg/kg/day to about 20 μg/kg/day, about 14 μg/kg/day to about 15 μg/kg/day, about 15 μg/kg/day to about 20 μg/kg/day, about 10 μg/kg/day to about 15 μg/kg/day, about 11 μg/kg/day to about 15 μg/kg/day, about 12 μg/kg/day to about 15 μg/kg/day, about 13 μg/kg/day to about 15 μg/kg/day, about 14 μg/kg/day to about 15 μg/kg/day, about 16 μg/kg/day to about 20 μg/kg/day, about 17 μg/kg/day to about 20 μg/kg/day, about 18 μg/kg/day to about 20 μg/kg/day, about 0.01 μg/kg/day, about 0.05 μg/kg/day, about 0.1 μg/kg/day, about 0.5 μg/kg/day, about 1 μg/kg/day, about 2 μg/kg/day, about 3 μg/kg/day, about 4 μg/kg/day, about 5 μg/kg/day, about 6 μg/kg/day, about 7 μg/kg/day, about 8 μg/kg/day, about 9 μg/kg/day, about 10 μg/kg/day, about 11 μg/kg/day, about 12 μg/kg/day, about 13 μg/kg/day, about 14 μg/kg/day, about 15 μg/kg/day, about 16 μg/kg/day, about 17 μg/kg/day, about 18 μg/kg/day, about 19 μg/kg/day, or about 20 μg/kg/day, of the encapsulated cells as provided herein.

As described herein, the encapsulated cells producing the recombinant native human IL-2 can be used to create memory immunity against a tumor. Thus, in some embodiments, the methods provided herein can be used to prevent or reduce the probability of a tumor recurring either at the initial site of the tumor or a site that is distal to the origin of the tumor. In some embodiments, the tumor is a pancreatic tumor. In some embodiments, the methods of treating a tumor by generating (inducing) memory immunity comprise implanting a pharmaceutical composition comprising a population of encapsulated cells as provided for herein.

In some embodiments, methods of selectively activating CD8 and/or CD4 positive effector T cells are provided. Without being bound by any particular theory, the CD8 positive and/or CD4 positive effector cells are activated and trigger an immune response against the tumor. This can be initiated or enhanced by the secretion of native human IL-2 in the IP space from the encapsulated cells that are provided for herein. In some embodiments, the methods comprise implanting a pharmaceutical composition comprising a population of encapsulated cells as provided for herein.

In some embodiments, the effector T cells are selectively activated and expanded as compared to Tregs (CD4+CD25+FOXp3+). In some embodiments, the effector T cells (e.g., CD8 and/or CD4 positive T cells) are selectively activated and expanded at least 1, 2, 3, 4, or 5 times as compared to Tregs.

Exemplary cancers that can be treated by the methods provided for herein include, but are not limited to, carcinoma, lymphoma, blastoma, sarcoma, and leukemia or lymphoid malignancies. In an embodiment, the cancer affects a system of the body, e.g., the nervous system (e.g., peripheral nervous system (PNS) or central nervous system (CNS)), vascular system, skeletal system, respiratory system, endocrine system, lymph system, reproductive system, or gastrointestinal tract. In some embodiments, cancer affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Other examples of cancers include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

In some embodiments, the implantable construct (encapsulated cells) described herein may be used in a method to modulate (e.g., upregulate) the immune response in a subject. For example, upon administration to a subject, the implantable construct (or an antigenic and/or therapeutic agent disposed within) may modulate (e.g., upregulate) the level of a component of the immune system in a subject (e.g., increasing the level or decreasing the level of an immune system component). Exemplary immune system components that may be modulated by an implantable construct or related method described herein include stem cells (hematopoietic stem cells), NK cells, T cells (e.g., an adaptive T cell (e.g., a helper T cell, a cytotoxic T cell, memory T cell, or regulatory T cell) or an innate-like T cell (e.g., natural killer T cell, mucosal-associated invariant T cell, or gamma delta T cell), B cells, an antibody or fragment thereof, or other another component. In an embodiment, the modulation comprises increasing or decreasing the activation of a T cell or other immune system component (e.g., by about 5%, 10%, 15%, 20%, 25%, 30%, 35%, 40%, 45%, 50%, 55%, 60%, 65%. 70%, 75%, 80%, 85%, 90%, 95%, 99%, or more compared with a control). In some embodiments, the encapsulated cells (implantable construct) can be used to activate CD4 positive and/or CD8 positive immune cells.

The implantable construct described herein may be used to modulate the immune response in a subject for a specific period of time. For example, administration of the implantable construct (or an antigenic and/or therapeutic agent disposed within) may activate the immune response (e.g., by increase in the level of an immune system component) in a subject for at least 1 hour, 2 hours, 3 hours, 4 hours, 6 hours, 8 hours, 10 hours, 12 hours, 16 hours, 20 hours, 1 day, 1.5 days, 2 days, 3 days, 4 days, 5 days, 6 days, 1 week, 1.5 weeks, 2 weeks, 3 weeks, 4 weeks, 5 weeks, 6 weeks, 2 months, 2.5 months, 3 months, 4 months, 5 months, 6 months, or longer. In an embodiment, administration of the implantable construct activates the immune response (e.g., by increase in the level of an immune system component) in a subject between 1 hour and 1 month, 1 hour and 3 weeks, 1 hour and 2 weeks, 1 hour and 1 week, 6 hours and 1 week, or 6 hours and 3 days. In an embodiment, implantation of the implantable construct (e.g., an implantable construct described herein) results in upregulation of T cells in a subject, e.g., as measured by a blood test, for at least 1 day.

The implantable constructs described herein may further comprise an additional pharmaceutical agent, such as an anti-proliferative agent, anti-cancer agent, anti-inflammatory agent, an immunomodulatory agent, or a pain-relieving agent, e.g., for use in combination therapy. The additional pharmaceutical agent may be disposed in or on the implantable construct or may be produced by a cell disposed in or on the implantable construct. In an embodiment, the additional pharmaceutical agent is small molecule, a protein, a peptide, a nucleic acid, an oligosaccharide, or other agent.

In an embodiment, the additional pharmaceutical agent is an anti-cancer agent. In some embodiments, the anti-cancer agent is a small molecule, a kinase inhibitor, an alkylating agent, a vascular disrupting agent, a microtubule targeting agent, a mitotic inhibitor, a topoisomerase inhibitor, an anti-angiogenic agent, or an anti-metabolite. In an embodiment, the anti-cancer agent is a taxane (e.g., paclitaxel, docetaxel, larotaxel or cabazitaxel). In an embodiment, the anti-cancer agent is an anthracycline (e.g., doxorubicin). In some embodiments, the anti-cancer agent is a platinum-based agent (e.g., cisplatin or oxaliplatin). In some embodiments, the anti-cancer agent is a pyrimidine analog (e.g., gemcitabine). In some embodiments, the anti-cancer agent is chosen from camptothecin, irinotecan, rapamycin, FK506, 5-FU, leucovorin, or a combination thereof. In other embodiments, the anti-cancer agent is a protein biologic (e.g., an antibody molecule), or a nucleic acid therapy (e.g., an antisense or inhibitory double stranded RNA molecule).

In an embodiment, the additional pharmaceutical agent is an immunomodulatory agent, e.g., one or more of an activator of a costimulatory molecule, an inhibitor of an immune checkpoint molecule, or an anti-inflammatory agent. In an embodiment, the immunomodulatory agent is an inhibitor of an immune checkpoint molecule (e.g., an inhibitor of PD-1, PD-L1, LAG-3, TIM-3 or CTLA4, or any combination thereof). In some embodiments, the immunomodulatory agent is a cancer vaccine.

In some embodiments, the immunomodulatory agent is an inhibitor of PD-1, PD-L1, PD-L2, CTLA4, TIM3, LAG3, VISTA, BTLA, TIGIT, LAIR1, CD73, CD160, 2B4 and/or TGFR beta. In one embodiment, the inhibitor of an immune checkpoint molecule inhibits PD-1, PD-L1, LAG-3, TIM-3 or CTLA4, or any combination thereof. Inhibition of an inhibitory molecule can be performed at the DNA, RNA or protein level. In some embodiments, an inhibitory nucleic acid (e.g., a dsRNA, siRNA or shRNA), can be used to inhibit expression of an inhibitory molecule. In other embodiments, the inhibitor of an inhibitory signal is, a polypeptide e.g., a soluble ligand (e.g., PD-1-Ig or CTLA-4 Ig), or an antibody or antigen-binding fragment thereof, that binds to the inhibitory molecule; e.g., an antibody or fragment thereof that binds to PD-1, PD-L1, PD-L2, CTLA4, TIM3, LAG3, VISTA, BTLA, TIGIT, LAIR1, CD73, CD160, 2B4 and/or TGFR beta, or a combination thereof. In some embodiments, the immunomodulatory agent is an anti-inflammatory agent, e.g., an anti-inflammatory agent as described herein. In an embodiment, the anti-inflammatory agent is an agent that blocks, inhibits, or reduces inflammation or signaling from an inflammatory signaling pathway. In an embodiment, the anti-inflammatory agent inhibits or reduces the activity of one or more of any of the following an immune component of the subject. In an embodiment, the anti-inflammatory agent is an IL-1 or IL-1 receptor antagonist, such as anakinra, rilonacept, or canakinumab. In an embodiment, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In an embodiment, the anti-inflammatory agent is a TNF-α antagonist, e.g., an anti-TNF-α antibody, such as infliximab (REMICADE®), golimumab (SIMPONI®), adalimumab (HUMIRA®), certolizumab pegol (CIMZIA®) or etanercept. In one embodiment, the anti-inflammatory agent is a corticosteroid, e.g., as described herein.

### F. Compositions and Administrations of Implantable Constructs

The present disclosure also provides pharmaceutical compositions comprising an implantable construct as provided for herein and optionally a pharmaceutically acceptable excipient. In some embodiments, the implantable construct is provided in an effective amount in the pharmaceutical composition. In some embodiments, the effective amount is a therapeutically effective amount. In some embodiments, the effective amount is a prophylactically effective amount. In some embodiments, the effective amount is an amount that produces an effective amount of native human IL-2.

Pharmaceutical compositions described herein can be prepared by any method known in the art of pharmacology. In general, such preparatory methods include the steps of bringing the implantable construct into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the implantable construct may be generally equal to the dosage of the antigenic and/or therapeutic agent which would be administered to a subject and/or a convenient fraction of such a dosage such as, for example, one-half or one-third of such a dosage.

In some embodiments, the pharmaceutical compositions are frozen or cryopreserved. In some embodiments, the pharmaceutical compositions are not frozen or not cryopreserved.

Relative amounts of the implantable construct, the pharmaceutically acceptable excipient, and/or any additional ingredients in a pharmaceutical composition of the disclosure will vary, depending upon the identity, size, and/or condition of the subject treated and further depending upon the route by which the composition is to be administered. By way of example, the composition may comprise between 0.1% and 100% (w/w) of any component.

The implantable construct and a pharmaceutical composition thereof may be administered or implanted orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally. In an embodiment, the implantable construct is injected subcutaneously. In an embodiment, the implantable construct is injected into the intraperitoneal space. In an embodiment, the implantable construct is injected into the intraperitoneal space. In an embodiment, the implantable constructed is delivered to the subject using a device, e.g., a cannula or catheter.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For ophthalmic use, provided compounds, compositions, and devices may be formulated as micronized suspensions or in an ointment such as petrolatum.

In an embodiment, the release of an antigenic, therapeutic, or additional pharmaceutical agent is released in a sustained fashion. In order to prolong the effect of a particular agent, it is often desirable to slow the absorption of the agent from injection. This can be accomplished by the use of a liquid suspension of crystalline or amorphous material with poor water solubility. The rate of absorption of the agent then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The implantable constructs provided herein are typically formulated in dosage unit form, e.g., single unit dosage form, for ease of administration and uniformity of dosage. It will be understood, however, that the total daily usage of the compositions of the present disclosure will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific therapeutic agent employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

The therapeutic agent administered may be at dosage levels sufficient to deliver from about 0.00001 mg/kg to about 100 mg/kg, from about 0.0001 mg/kg to about 100 mg/kg, from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 0.001 mg/kg to about 1 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

The constructs (e.g., encapsulated cells) can be prepared according to any known method. For example, in some embodiments, methods of preparing encapsulated cells producing a recombinant protein are provided. In some embodiments, the methods comprise feeding through a coaxial needle a first composition comprising a polymeric hydrogel and a second composition comprising cells to be encapsulated suspended in a polymeric hydrogel to drop into a crosslinking solution to form the encapsulated cells, wherein the crosslinking solution comprises a sugar alcohol, a buffer, a metal salt, and a surfactant. In some embodiments, the cells to be encapsulated comprise an oligonucleotide molecule encoding native human IL-2. In some embodiments, the oligonucleotide encoding native human IL-2 comprises a sequence of SEQ ID NO: 1. In some embodiments, the cell produces recombinant native human IL-2 protein. In some embodiments, the IL-2 protein is formed from an amino acid sequence of SEQ ID NO: 2.

The cells can be any type of cell. In some embodiments, the cell is a mammalian cell. In some embodiments, the cell is an epithelial cell. In some embodiments, the cell is a RPE cell. In some embodiments, the cell is a ARPE-19 cell, ARPE-19-SEAP-2-neo cell, RPE-J cell, and hTERT RPE-1 cell. In some embodiments, the cell is an engineered RPE cell. In some embodiments, the engineered cell is derived from the ARPE-19 cell line. In some embodiments, the cell is as provided herein. In some embodiments, the surfactant is TWEEN 20 (polysorbate 20). In some embodiments, the buffer is HEPES buffer. In some embodiments, the sugar alcohol is mannitol. In some embodiments, the metal salt is barium chloride.

In some embodiments, the method comprises washing the encapsulated cells produced according to the methods provided for herein in a buffer solution produced. In some embodiments, the washing step removes substantially all or all of the free barium or barium chloride.

In some embodiments, the encapsulated cells prepared according to the methods provided herein are stored in a storage buffer, such as DMEM/F12 cell culture media. In some embodiments, the stored cells retain viability for at least 10, 20, or 30 days. In some embodiments, the storage buffer is substantially free or free of plasmalyte buffer.

Also provided for herein, are a population of encapsulated cells prepared according to a method as provided for herein.

In some embodiments, a suspension of encapsulated cells is provided. In some embodiments, the suspension comprises a population of encapsulated cells as provided for herein. In some embodiments, the encapsulated cells are encapsulated by a polymeric hydrogel, and the suspension comprises a crosslinking solution that comprises a sugar alcohol, a buffer, a metal salt, and a surfactant. In some embodiments, the cells are ARPE-19 cells. In some embodiments, the surfactant is TWEEN 20 (polysorbate 20). In some embodiments, the buffer is HEPES buffer. In some embodiments, the sugar alcohol is mannitol. In some embodiments, the metal salt is barium chloride.

In some embodiments, suspensions of encapsulated cells are provided, wherein the suspension comprises a population of encapsulated cells as provided for herein, wherein the encapsulated cells are encapsulated by a polymeric hydrogel, and a storage buffer, such as DMEM/F12 cell culture media. In some embodiments, the suspended encapsulated cells retain viability for at least 10, 20, or 30 days.

In some embodiments, the suspension provided for herein are substantially free or free of plasmalyte buffer.

### ENUMERATED EMBODIMENTS

1. A population of encapsulated cells comprising an oligonucleotide molecule encoding native human IL-2.
2. The population of encapsulated cells of embodiment 1, wherein the oligonucleotide encoding native human IL-2 comprises a sequence of SEQ ID NO: 1 or codon optimized oligonucleotide sequence thereof.
3. The population of encapsulated cells of embodiment 2, wherein the codon-optimized oligonucleotide encoding native human IL-2 comprises a sequence having at least 90%, 91%, 92%, 93%, 94%, 95%, 96%, 97%, 98%, or 99% sequence identity to SEQ ID NO: 3.
4. The population of encapsulated cells of embodiment 3, wherein the codon-optimized oligonucleotide encoding native human IL-2 comprises a sequence of SEQ ID NO: 3.
5. The population of encapsulated cells of any one of embodiments 1-4, wherein the cell produces recombinant native human IL-2 protein.
6. The population of encapsulated cells of any one of embodiments 1-5, wherein the recombinant native human IL-2 protein expressed by the cells comprises the amino acid sequence of SEQ ID NO: 2.
7. The population of encapsulated cells of any one of embodiments 1-6, wherein the population of cell produces about 1 to about 10, about 1 to about 5, or about 2 to about 4 PCD (picograms/cell/day) of recombinant native human IL-2.
8. The population of encapsulated cells of any one of embodiments 1-7, wherein the cells are retinal pigment epithelial cells.
9. The population of encapsulated cells of any one of embodiments 1-8, wherein the cells are ARPE-19 cells, ARPE-19-SEAP-2-neo cells, RPE-J cells, hTERT RPE-1 cells, or any combination thereof.
10. The population of encapsulated cells of any one of embodiments 1-9, wherein the cells are encapsulated with a polymeric hydrogel.
11. The population of encapsulated cells of any one of embodiments 1-10, wherein the polymeric hydrogel comprises chitosan, cellulose, hyaluronic acid, or alginate.
12. The population of encapsulated cells of any one of embodiments 1-11, wherein the polymeric hydrogel comprises alginate.
13. The population of encapsulated cells of any one of embodiments 1-12, wherein the alginate comprises SLG20.
14. The population of encapsulated cells of any one of embodiments 1-13, wherein the encapsulated cells remain viable for at least 15, 20, 25, or 28 days.
15. The population of encapsulated cells of any one of embodiments 1-13, wherein the encapsulated cells do not proliferate.
16. The population of encapsulated cells of any one of embodiments 1-13, wherein the encapsulated cells produced a sustained amount of recombinant native IL-2 for at least 5, 10, 15, 20, or 24 hours.
17. The population of encapsulated cells of any one of embodiments 1-13, wherein the encapsulated cells can produce a sustained amount of recombinant native IL-2 for up to 30 days.
18. A pharmaceutical composition comprising the population of encapsulated cells of any one of embodiments 1-17.
19. A method of treating a tumor in a subject, the method comprising implanting in the intraperitoneal space of the subject a pharmaceutical composition comprising a plurality of encapsulated cells of any one of embodiments 1-17 to the subject to treat the cancer.
20. The method of embodiment 19, wherein the subject has a pancreatic tumor.
21. The method of embodiments 18 or 19, wherein the concentration of recombinant native human IL-2 in the intraperitoneal space at day 5 post implantation is at least 5000 pg/ml, 10000 pg/ml, 15000 pg/ml, 20000 pg/ml, 50000 pg/ml, 100000 pg/ml, or 150000 pg/ml.
22. The method of any one of embodiments 19-21, wherein the concentration of the recombinant native human IL-2 in the intraperitoneal space is at least 100× greater than the concentration of the recombinant native human IL-2 in the blood of the subject.
23. The method of any one of embodiments 19-22, wherein the concentration of the recombinant native human IL-2 in the blood of the subject is substantially undetectable 5 days after implantation.
24. The method of embodiment 19, wherein the concentration of the recombinant native human IL-2 in the blood of the subject is substantially undetectable 5 days after implantation and is at least 5000 pg/ml, 10000 pg/ml, 15000 pg/ml, 20000 pg/ml, 50000 pg/ml, 100000 pg/ml, or 150000 pg/ml in the intraperitoneal space of the subject.
25. The method of embodiment 19, wherein the recombinant native IL-2 protein is detectable in the intraperitoneal space of the subject at least 1, 4, 7, 14, 21, or 30 days post implantation.
26. The method of any one of embodiments 19-25, wherein the subject is administered about 0.01 μg/kg/day to about 20 μg/kg/day, about 0.1 μg/kg/day to about 20 μg/kg/day, about 1 μg/kg/day to about 20 μg/kg/day, about 2 μg/kg/day to about 20 μg/kg/day, about 5 μg/kg/day to about 20 μg/kg/day, about 7.5 to about 20 μg/kg/day, about 9 μg/kg/day to about 20 μg/kg/day, about 10 μg/kg/day to about 20 μg/kg/day, about 11 μg/kg/day to about 20 μg/kg/day, about 12 μg/kg/day to about 20 μg/kg/day, about 13 μg/kg/day to about 20 μg/kg/day, about 14 μg/kg/day to about 15 μg/kg/day, about 15 μg/kg/day to about 20 μg/kg/day, about 10 μg/kg/day to about 15 μg/kg/day, about 11 μg/kg/day to about 15 μg/kg/day, about 12 μg/kg/day to about 15 μg/kg/day, about 13 μg/kg/day to about 15 μg/kg/day, about 14 μg/kg/day to about 15 μg/kg/day, about 16 μg/kg/day to about 20 μg/kg/day, about 17 μg/kg/day to about 20 μg/kg/day, about 18 μg/kg/day to about 20 μg/kg/day, about 0.01 μg/kg/day, about 0.1 μg/kg/day, about 1 μg/kg/day, about 2 μg/kg/day, about 3 μg/kg/day, about 4 μg/kg/day, about 5 μg/kg/day, about 6 μg/kg/day, about 7 μg/kg/day, about 8 μg/kg/day, about 9 μg/kg/day, about 10 μg/kg/day, about 11 μg/kg/day, about 12 μg/kg/day, about 13 μg/kg/day, about 14 μg/kg/day, about 15 μg/kg/day, about ′6 μg/kg/day, about 17 μg/kg/day, about 18 μg/kg/day, about 19 μg/kg/day, or about 20 μg/kg/day, of the encapsulated cells of any one of embodiments 1-17.
27. A method of treating a tumor in a subject by generating memory immunity, the method comprising implanting a pharmaceutical composition comprising the population of encapsulated cells of any one of embodiments 1-17.
28. The method of embodiment 27, wherein the subject has a pancreatic tumor.
29. A method of selectively activating CD8 positive effector T cells in a subject, the method comprising implanting in the subject a pharmaceutical composition comprising a population of encapsulated cells of any one of embodiments 1-17.
30. The method of embodiment 29, wherein the effector T cells are selectively activated and expanded as compared to Tregs (CD4+CD25+FOXp3+).
31. The method of embodiment 30, wherein the effector T cells are selectively activated and expanded at least 1, 2, 3, 4, or 5 times as compared to Tregs.
32. A method of providing systemic treatment to a subject with cancer, the method comprising implanting in the intraperitoneal space of the subject a pharmaceutical composition comprising a plurality of encapsulated cells of any one of embodiments 1-17, whereby the pharmaceutical composition stimulates the activation of immune cells in the intraperitoneal space and the activated immune cells migrate to a region of the subject that is distal to the intraperitoneal space to treat the cancer systemically in the subject.
33. A method of providing systemic treatment to a subject with cancer, the method comprising implanting in the intraperitoneal space of the subject a pharmaceutical composition comprising a plurality of encapsulated cells of any one of embodiments 1-17, whereby the pharmaceutical composition activates immune cells and the activated immune cells migrate out of the intraperitoneal space to treat the cancer in the subject.
34. The method of embodiments 32 or 33, wherein the subject has fewer side effects as compared to a subject that is administered the pharmaceutical composition systemically, such as intravenously.
35. The method of any one of embodiments 32-34, wherein the activated immune cells are CD8 positive effector T cells.
36. The method of any one of embodiments 32-35, wherein the effector T cells are selectively activated and expanded at least 1, 2, 3, 4, or 5 times as compared to Tregs in the intraperitoneal space.
37. The method of any one of embodiments 32-36, wherein the effector T cells are selectively activated and expanded at least 1, 2, 3, 4, or 5 times as compared to Tregs systemically.
38. The method of any one of embodiments 32-37, wherein the concentration of native human IL-2 in the intraperitoneal space at day 5 post implantation is at least 5000 pg/ml, 10000 pg/ml, 15000 pg/ml, 20000 pg/ml, 50000 pg/ml, 100000 pg/ml, or 150000 pg/ml.
39. The method of any one of embodiments 32-38, wherein the concentration of the native human IL-2 in the intraperitoneal space is at least 100× greater than the concentration of the native human IL-2 in the blood of the subject.
40. The method of any one of embodiments 32-39, wherein the concentration of the recombinant native human IL-2 in the blood of the subject is substantially undetectable 5 days after implantation.
41. The method of any one of embodiments 32-40, wherein the concentration of the recombinant native human IL-2 in the blood of the subject is substantially undetectable 5 days after implantation and is at least 5000 pg/ml, 10000 pg/ml, 15000 pg/ml, 20000 pg/ml, 50000 pg/ml, 100000 pg/ml, or 150000 pg/ml in the intraperitoneal space of the subject.
42. The method of any one of embodiments 32-41, wherein the recombinant native IL-2 protein is detectable in the intraperitoneal space of the subject at least 1, 4, 7, 14, 21, or 30 days post implantation. 43. A method of preparing encapsulated cells producing a recombinant protein, the method comprising:
   feeding through a coaxial needle a first composition comprising a polymeric hydrogel and a second composition comprising cells to be encapsulated suspended in a polymeric hydrogel to drop into a crosslinking solution to form the encapsulated cells, wherein the crosslinking solution comprises a sugar alcohol, a buffer, a metal salt, and a surfactant.
44. The method of embodiment 43, wherein the cells to be encapsulated comprise a heterologous oligonucleotide molecule encoding recombinant native human IL-2.
45. The method of embodiments 43 or 44, wherein the recombinant oligonucleotide encoding recombinant native human IL-2 comprises a sequence of SEQ ID NO: 1 or a codon optimized version thereof.
46. The population of encapsulated cells of embodiment 45, wherein the codon-optimized oligonucleotide encoding the recombinant native human IL-2 comprises a sequence having at least 90%, 91%, 92%, 93%, 94%, 95%, 96%, 97%, 98%, or 99% sequence identity to SEQ ID NO: 3.
47. The population of encapsulated cells of embodiment 45, wherein the codon-optimized oligonucleotide encoding the recombinant native human IL-2 comprises a sequence of SEQ ID NO: 3.
48. The method of any one of embodiments 43-47, wherein the cell produces recombinant native human IL-2 protein comprising an amino acid sequence of SEQ ID NO: SEQ ID NO: 2.
49. The method of any one of embodiments 43-48, the cells are ARPE-19 cells, ARPE-19-SEAP-2-neo cells, RPE-J cells, hTERT RPE-1 cells, or any combination thereof.
50. The method of any one of embodiments 43-49, wherein the surfactant is TWEEN 20 (polysorbate 20).
51. The method of any one of embodiments 43-50, wherein the buffer is HEPES buffer.
52. The method of any one of embodiments 43-51, wherein the sugar alcohol is mannitol.
53. The method of any one of embodiments 43-52, wherein the metal salt is barium chloride.
54. The method of any one of embodiments 43-53, wherein the method further comprises washing the encapsulated cells in a buffer solution.
55. The method of any one of embodiments 43-54, wherein the method further comprises storing the encapsulated cells in a storage buffer
56. The method of embodiment 55, wherein the storage buffer comprises DMEM/F12 cell culture media.
57. The method of embodiments 55 or 56, wherein the stored cells retain viability for at least 30 days.
58. The method of any one of embodiments 55-57, wherein the storage buffer is substantially free or free of plasmalyte buffer. 59. A population of encapsulated cells prepared according to a method of any one of embodiments 43-58.
60. A suspension of encapsulated cells, wherein the suspension comprises a population of encapsulated cells of any one of embodiments 1-17, wherein the encapsulated cells are encapsulated by a polymeric hydrogel, and the suspension comprises a crosslinking solution that comprises a sugar alcohol, a buffer, a metal salt, and a surfactant.
61. The suspension of embodiment 60, wherein the cells are ARPE-19 cells, ARPE-19-SEAP-2-neo cells, RPE-J cells, hTERT RPE-1 cells, or any combination thereof.
62. The suspension of embodiments 60 and 61, wherein the surfactant is TWEEN 20 (polysorbate 20).
63. The suspension of any one of embodiments 60-62, wherein the buffer is HEPES buffer.
64. The suspension of any one of embodiments 60-63, wherein the sugar alcohol is mannitol.
65. The suspension of any one of embodiments 60-64, wherein the metal salt is barium chloride.
66. A suspension of encapsulated cells, wherein the suspension comprises a population of encapsulated cells of any one of embodiments 1-17, a storage buffer, wherein the encapsulated cells are encapsulated by a polymeric hydrogel.
67. The suspension of embodiment 66, wherein the suspended encapsulated cells retain viability for at least 30 days.
68. The suspension of any one of embodiments 66 or 67, wherein the suspension buffer is substantially free or free of plasmalyte buffer.

### G. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain alike or similar result without departing from the spirit and scope of the disclosure.

### Example 1—Native hIL-2 is More Potent than Recombinant hIL-2

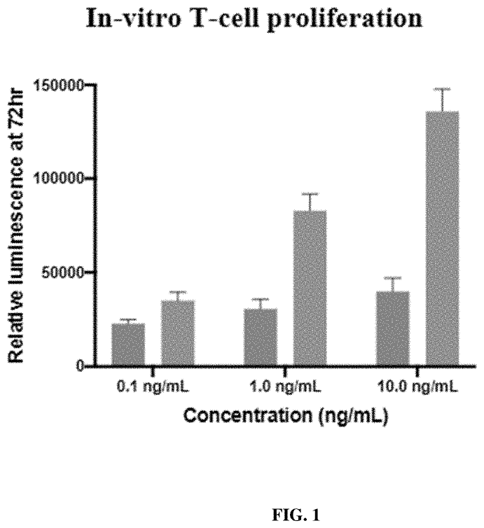

Encapsulated cells expressing hIL-2 were manufactured by encapsulating genetically modified cells that produce hIL-2 in SLG20. An EasySep T Cell Isolation Kit (StemCellTechnologies) and associated reagents and devices were used to isolate T cells from C57BL6 mouse spleens (Jackson Labs or Charles River Laboratories). Cells were plated in 96 well plates at 10,000 cells/well in 100 ul RPMI 1640. Cells were supplemented with 0.1, 1 or 10 ng/mL of cell produced native IL-2 (conditioned media the cells) or recombinant human IL-2 (Teceleukin). 72 hours after incubation at 37° C., cell proliferation was measured using CellTiter-Glo Luminescent Cell Viability Assay (Promega). hIL-2 produced by the cells resulted in T-Cell Proliferation. FIG. 1 shows the recombinant IL-2 (left bar) and the native human IL-2 which can also be referred to as “recombinant native IL-2”, produced by the encapsulated cells (right bar). At all three hIL-2 concentrations tested in vitro, the native human IL-2 produced by the encapsulated cells was more potent than purified human recombinant IL-2. These results demonstrate that the encapsulated cells expressing the native IL-2 are more potent and should have a greater therapeutic effect as compared to the use of recombinant IL-2 that is injected into the IP space of a subject.

### Example 2—Dose-Dependent hIL-2 Concentration in Intraperitoneal Cavity

To test the ability to tune the local concentration of native, human IL-2 (hIL-2) delivered, the number of capsules administered within a given dose was varied. Three doses of encapsulated cells expressing native human IL-2 were implanted in the intraperitoneal space of non-human primates and the IP fluid and blood concentration of hIL-2 was assayed via ELISA. The IP concentration of hIL-2 increased with the number of encapsulated cells delivered and regardless of the dose, hIL-2 levels measured in the IP fluid were higher than the levels found in the blood, which remained well below the toxic threshold. Briefly, encapsulated cells were manufactured by encapsulating genetically modified ARPE-19 cells that produce native hIL-2. The cells were administered at different doses (132 capsule/kg=5 μg hIL2/kg, 264 capsule/kg=9.2 μg hIL2/kg, 528 capsule/kg=18.3 μg hIL2/kg), once to the intraperitoneal space of the cynomolgus monkey NHP (n=1 per group) by a laparoscopic procedure. Blood and IP fluid were collected after administration of the capsules and hIl-2 concentration was determined in NHP samples using an hIL-2 ELISA assay. The levels of detectable hIL-2 in the blood peak on Day 1 after administration and decline to undetectable levels by Day 5. The ratio of IP to blood of IL-2 was at least 150× at all doses by day 6. The levels of hIL-2 in the IP fluid demonstrated a dose-dependent increase at day 5 and reached therapeutic level for each dose tested. Furthermore, the encapsulated cells induced an increased in proliferation of CD8+ and CD4+ T cells in the peritoneal cavity cells (data not shown) in NHP. Together, the hIL-2 levels measured in all monkeys follow the same trend of detectable levels in the IP fluid at day 5. These findings demonstrate the ability to increase the local concentration of hIL-2 in the IP space without significantly increasing systemic exposure to hIL-2, thereby reducing toxic effects of hIL-2.

### Example 3—Administration of Encapsulated Cells Expressing IL-2 Protein Show Dose-Dependent Effect on ID8 Ovarian Tumors in Mice

To test the ability to tune local concentration of mIL-2 delivered in vivo and its effect on ovarian tumor suppression, the inventors varied the number of capsules within a given dose. Four doses of capsules comprising encapsulated RPE cells expressing IL-2 were administered in the intraperitoneal space six days after ID8 F-Luc cells were injected into mice. Encapsulated RPE cells expressing IL-2 showed a fast and dose dependent tumor response as monotherapy in syngeneic ID8 Ovarian Mouse Model. ID8 F-Luc cells were injected into C57BL/6 albino mice IP space at a cell density of 1×10⁷/mouse. Six days post IP injections, animals were randomly stratified into groups and either subjected to a sham surgery or were surgically administered with one dose of either 10, 50, 100 or 200 murine capsules (each capsule contained approximately 3×10⁴ RPE-murine IL2 cells) and tracked with luminescent imaging for 30 days. After only six days of treatment, mice that received doses of 100 or 200 capsules exhibited a reduction in tumor burden that was 3.3× and 7.5×, respectively, greater than sham mice. After 30 days there was a significant reduction in tumor burden across all groups that received murine capsules. When compared to tumor burden measured in the sham mice, mice with 10, 50, 100 or 200 capsules showed 3.1×, 5×, 53× and 147×, respectively, less tumor burden. Murine capsules showed a fast and dose dependent tumor response as monotherapy in syngeneic ID8 Ovarian Mouse Model in mice.

### Example 4—Administration of Encapsulated Cells Expressing IL-2 Protein Induce Long Lasting Tumor Immunity

To test the ability of encapsulated RPE-expressing IL-2 confers protection from distal tumor rechallenge, the inventors tested whether such cells are sufficient to lead to the rejection of a tumor rechallenge in the absence of therapy. Mice that had undergone complete tumor regression following previous administration of RPE-expressing IL-2 were rechallenged with MC38 injected subcutaneously. Briefly, MC-38 F-Luc; 1×10⁶ cells suspended in HBSS were intraperitoneally injected to the lower right abdomen of C57BL6 Albino males and females mixed cohorts. 7 days post IP injections, animals were surgically administered with one dose of encapsulated cells expressing IL-2. 28 days later, mice that had undergone complete tumor regression following treatment were rechallenged with 5×10⁵ MC38 cells. MC-38 tumor cells suspended in HBSS and were injected subcutaneously into the rear flank of C57BL6 Albino male and female mice. Tumor-naïve C57ALB mice (n=5) injected subcutaneously into the rear flank with MC-38 tumor cells suspended in HBSS were used as control. After nearly two weeks, none of the five previously treated mice developed a subcutaneous tumor while five out of the eight naïve mice developed visible tumor just four days post tumor injection. These data demonstrate that the encapsulated cells expressing native IL-2 yielded system wide protective memory immunity against tumor cells, which was a surprising and unexpected result.

### Example 5—Encapsulated Cells are Non-Tumorigenic

Implantable cells can become tumorigenic, thus it is important to demonstrate the inability of the encapsulated cells to become tumorigenic or divide uncontrollably. The cells that were used, ARPE-19 cells, were selected because it is non-tumorigenic, displays contact-inhibited growth characteristics in 2D culture, is amendable to genetic modification, and has shown to be safe in prior clinical studies. In order to assess ARPE-19 cell fate after encapsulation various assays were performed to analyze the viability and proliferation of the encapsulated ARPE-19 cells within the capsules. These studies demonstrated that encapsulated ARPE-19 cells exhibited the desired characteristics including viability in culture for at least 4 weeks, expansion in 2D culture, and contact inhibition upon encapsulation, preventing further cell growth within the capsules. Briefly, the LIVE/DEAD Viability/Cytotoxicity Kit was used to assess ARPE-19 viability within encapsulation over time in vitro to quickly discriminate live cells from dead cells by simultaneously staining with green-fluorescent stain to indicate intracellular esterase activity (live cells) and red-fluorescent ethidium homodimer-1 (dead cells) to indicate loss of plasma membrane integrity. Proliferation of the cells within the capsules was measured using Click-iT EdU imaging kit. A 1×working solution of EdU diluted in media was added to each capsule. At 0, 24, 72 hours, and 7 days, the media was removed and capsules were fixed in 4% PFA. Capsules were washed with 3% BSA, and permeabilized using 0.5% Triton X-100. To stain the cells, 0.2 ml of the Click-iT reaction cocktail was added to each capsule and incubated for 30 minutes. The capsules were subsequently washed with 3% BSA. Lastly, cell nuclei were stained using Hoechst 33343 (NucBlu). Capsules were imaged using an EVOS XL microscope at 4× magnification.

First, the inventors assessed the viability of the encapsulated ARPE-19 using live/dead stain by fluorescence microscopy. The observed no differences in the number of encapsulated viable ARPE-19 cells in vitro over the 28-day period. Next, the inventors investigated the proliferation status of ARPE-19 cells. An in vitro assessment of the capsules was conducted to compare proliferation of encapsulated ARPE cells with cells known to exhibit continued in vitro proliferation within alginate hydrogels (HEK cells). ARPE cells (or HEK cells as a control) were encapsulated into 1.5 mm alginate capsules and imaged in vitro over time for up to 7 days. At discreet time points, capsules were pulled from the main population at random and assayed qualitatively using DAPI staining to visualize the cells within the capsules and EdU (GFP) as a proliferation marker. Images were taken at 4× magnification. Only the HEK cells stained with the GFP marker, confirming that the ARPE-19 cells do not proliferate after encapsulation within the alginate hydrogels (data not shown). This observation was confirmed using PCR analysis. In conclusion, in vitro the encapsulated ARPE-19 remained viable for at least 28 days. The encapsulated ARPE-19 cell line could be expanded in 2D culture but exhibited contact inhibition upon encapsulation and thus did not continue expanding inside of the capsules. This feature is critical for regulating the dose of cytokine secretion per capsule post administration.

### Example 6—Preparation of Encapsulated Cells

Polyclonal ARPE-19 cells were expanded and transfected using a lipofectamine protocol with a ratio of 5:1 (transposase:transposon) to create cells expressing human native IL-2. Transfected cells were cultured are plated at 0.5 cells/well for single cell outgrowth. IL-2 production of the selected clone was about −3.8 PCD (picograms/cell/day). The clone was expanded in cell flasks/stacks for up to two weeks before being harvested into a cell pellet and suspended in alginate (SLG20) for encapsulation. The encapsulation process comprises loading two syringes, one with SLG20, and one with the cell pellet (42 million cells/mL) suspended in alginate (SLG20). The syringes were fed into a coaxial needle through use of a power supply (electric current) allowing droplets to fall into a crosslinking bath, which contained mannitol, barium chloride, HEPES buffer and Tween 20, which was where the capsules take shape. Capsules were collected from the bath after sitting in the bath for 5 minutes and washed 8 times at a 1:25 ratio of capsules to HEPES buffer solution (2 minutes/wash) to help to help remove loosely bound barium. The encapsulated cells were stored in DMEM/F12 cell culture media at ambient temperature in biotainer bottle.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

1-68. (canceled)

69. A population of encapsulated cells comprising an oligonucleotide molecule encoding native human IL-2.

70. The population of encapsulated cells of claim 69, wherein the oligonucleotide encoding native human IL-2 comprises a sequence of SEQ ID NO: 1 or codon optimized oligonucleotide sequence thereof.

71. The population of encapsulated cells of claim 70, wherein the codon-optimized oligonucleotide encoding native human IL-2 comprises a sequence having at least 90%, 91%, 92%, 93%, 94%, 95%, 96%, 97%, 98%, 99%, or 100% sequence identity to SEQ ID NO: 3.

72. The population of encapsulated cells of claim 69, wherein the cell produces recombinant native human IL-2 protein.

73. The population of encapsulated cells of claim 72, wherein the recombinant native human IL-2 protein expressed by the cells comprises the amino acid sequence of SEQ ID NO: 2.

74. The population of encapsulated cells of claim 69, wherein the population of cell produces about 1 to about 10, about 1 to about 5, or about 2 to about 4 PCD (picograms/cell/day) of recombinant native human IL-2.

75. The population of encapsulated cells of claim 69, wherein the cells are retinal pigment epithelial cells.

76. The population of encapsulated cells of claim 69, wherein the cells are encapsulated with a polymeric hydrogel.

77. The population of encapsulated cells of claim 76, wherein the polymeric hydrogel comprises chitosan, cellulose, hyaluronic acid, or alginate.

78. The population of encapsulated cells of claim 77, wherein the alginate comprises SLG20.

79. The population of encapsulated cells claim 69, wherein the encapsulated cells remain viable for at least 15, 20, 25, or 28 days.

80. The population of encapsulated cells of claim 69, wherein the encapsulated cells do not proliferate.

81. A pharmaceutical composition comprising the population of encapsulated cells of claim 69.

82. A method of treating a tumor in a subject, the method comprising implanting in the intraperitoneal space of the subject a pharmaceutical composition comprising a plurality of encapsulated cells of claim 69 to the subject to treat the cancer.

83. The method of claim 82, wherein the concentration of recombinant native human IL-2 in the intraperitoneal space at day 5 post implantation is at least 5000 pg/ml, 10000 pg/ml, 15000 pg/ml, 20000 pg/ml, 50000 pg/ml, 100000 pg/ml, or 150000 pg/ml.

84. The method of claim 82, wherein the concentration of the recombinant native human IL-2 in the intraperitoneal space is at least 100× greater than the concentration of the recombinant native human IL-2 in the blood of the subject.

85. The method of claim 82, wherein the concentration of the recombinant native human IL-2 in the blood of the subject is substantially undetectable 5 days after implantation.

86. The method of claim 82, wherein the recombinant native IL-2 protein is detectable in the intraperitoneal space of the subject at least 1, 4, 7, 14, 21, or 30 days post implantation.

87. A method of:
treating a tumor in a subject by generating memory immunity, the method comprising implanting in the subject a pharmaceutical composition comprising the population of encapsulated cells of claim 69;
selectively activating CD8 positive effector T cells in a subject, the method comprising implanting in the subject a pharmaceutical composition comprising a population of encapsulated cells of claim 69;
providing systemic treatment to a subject with cancer, the method comprising implanting in the intraperitoneal space of the subject a pharmaceutical composition comprising a plurality of encapsulated cells of claim 69, whereby the pharmaceutical composition stimulates the activation of immune cells in the intraperitoneal space and the activated immune cells migrate to a region of the subject that is distal to the intraperitoneal space to treat the cancer systemically in the subject; or
providing systemic treatment to a subject with cancer, the method comprising implanting in the intraperitoneal space of the subject a pharmaceutical composition comprising a plurality of encapsulated cells of claim 69, whereby the pharmaceutical composition activates immune cells and the activated immune cells migrate out of the intraperitoneal space to treat the cancer in the subject.

88. A method of preparing encapsulated cells producing a recombinant protein, the method comprising:
feeding through a coaxial needle a first composition comprising a polymeric hydrogel and a second composition comprising cells to be encapsulated suspended in a polymeric hydrogel to drop into a crosslinking solution to form the encapsulated cells, wherein the crosslinking solution comprises a sugar alcohol, a buffer, a metal salt, and a surfactant.


SEQUENCE LISTING (6 sequences)

SEQ ID NO: 1 - DNA, 462 bp, synthetic construct
  misc_feature 1..462: note = synthetic oligonucleotide
  source 1..462: mol_type = other DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 2 - protein, 153 aa, synthetic construct
  REGION 1..153: note = synthetic oligonucleotide
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 3 - DNA, 462 bp, synthetic construct
  misc_feature 1..462: note = synthetic oligonucleotide
  source 1..462: mol_type = other DNA
atgtaccggatgcagctgctgtcctgcatcgcactgtccctcgccctggtgacaaattctgcccccacctcctccagcacaaaaaagacccagttgcagctggagcacctgctgctggatctgcagatgatcctgaatggcatcaataactacaaaaaccctaaactgaccagaatgctgacctttaaattttacatgcctaaaaaggcaaccgagctgaagcacctgcagtgcctggaagaggaactgaagcccctggaggaggtgctgaacctggcccagagcaagaactttcacctgcggccccgcgacctgatcagcaacatcaacgtgatcgtgctggagctgaagggcagtgaaaccacattcatgtgcgagtacgccgacgagaccgccacaatcgtggagttcctgaacagatggatcacattctgtcagtccatcattagcacactgacctaa

SEQ ID NO: 4 - DNA, 462 bp, synthetic construct
  misc_feature 1..462: note = synthetic oligonucleotide
  source 1..462: mol_type = other DNA
atgtaccgcatgcagctgctgagctgcatcgccctgagcctggccctggtgaccaacagcgcccccaccagcagcagcaccaagaagacccagctgcagctggagcacctgctgctggacctgcagatgatcctgaacggcatcaacaactacaagaaccccaagctgacccgcatgctgaccttcaagttctacatgcccaagaaggccaccgagctgaagcacctgcagtgcctggaggaggagctgaagcccctggaggaggtgctgaacctggcccagagcaagaacttccacctgcgcccccgcgacctgatcagcaacatcaacgtgatcgtgctggagctgaagggcagcgagaccaccttcatgtgcgagtacgccgacgagaccgccaccatcgtggagttcctgaaccgctggatcaccttctgccagagcatcatcagcaccctgacctaa

SEQ ID NO: 5 - DNA, 459 bp, synthetic construct
  misc_feature 1..459: note = synthetic oligonucleotide
  source 1..459: mol_type = other DNA
atgtataggatgcagctgctctcttgtatcgcgttgtctctggctttggtgactaactcagctcccacgtccagcagtaccaaaaagacccagctgcagctggaacatcttctgttggatctgcaaatgatactgaatgggatcaacaactataaaaacccaaaactgactagaatgctgactttcaagttctacatgcctaaaaaggcaacagaattgaagcaccttcagtgcctggaggaggagcttaagcccctggaggaggtgctgaatctggcccaaagtaagaattttcatctgcgacccagggatctgatcagtaatatcaatgtgatcgtcctggagctgaagggcagtgagaccacgtttatgtgtgaatacgcagacgaaaccgccactatcgttgaattcttgaacaggtggatcaccttttgtcagagtatcatcagcaccctcact

SEQ ID NO: 6 - DNA, 462 bp, synthetic construct
  misc_feature 1..462: note = synthetic oligonucleotide
  source 1..462: mol_type = other DNA
atgtacagaatgcagctgctgagctgcatcgccctgagcctggccctggtgaccaacagcgcccccacaagcagcagcaccaagaagacacagctgcagctggagcacctgctgctggacctgcagatgatcctgaacggcatcaacaactacaagaaccccaagctgacaagaatgctgaccttcaagttctacatgcccaagaaggccaccgagctgaagcacctgcagtgcctggaggaggagctgaagcccctggaagaggtgctgaacctggctcagagcaagaacttccacctgagacctagagacctgatcagcaacatcaacgtgatcgtgctggagctgaagggcagcgagaccaccttcatgtgcgagtacgccgacgagaccgccaccatcgtggagttcctgaacagatggatcaccttctgtcagagcatcatcagcaccctgacctga